(19) United States
(12) Patent Application Publication
(10) Pub. No.: US 2025/0073344 A1
(43) Pub. Date: Mar. 6, 2025

(54) METHODS OF TREATING MALIGNANT GLIOMAS

(71) Applicant: Targepeutics, Inc., Hummelstown, PA (US)

(72) Inventors: Randy Schrecengost, Lancaster, PA (US); Silvan B Lutkewitte, III, Hershey, PA (US)

(21) Appl. No.: 18/294,303

(22) PCT Filed: Nov. 10, 2022
(86) PCT No.: PCT/US2022/079654

§ 371 Date: Feb. 1, 2024

Related U.S. Application Data
(60) Provisional application No. 63/277,866, filed on Nov. 10, 2021.

(51) Int. Cl. A61K 47/64; A61K 38/16; A61P 35/00; G01N 33/68

(52) U.S. Cl. CPC A61K 47/642; A61K 38/164; A61P 35/00; G01N 33/6869; G01N 2333/46; G01N 2333/7155; G01N 2800/52

(57) ABSTRACT

Disclosed is a method of treating a subject having a malignant glioma comprising: administering by convection-enhanced delivery (CED) for a period of up to 96 hours a therapeutically effective amount of a mutagenized IL13 linked to a cytotoxin (cmIL13); wherein the malignant glioma expresses an interleukin 13 receptor α 2 (IL13Rα2). Also disclosed is a mutagenized IL13 for use in methods of treating a subject having a malignant glioma expressing IL13Rα2, wherein the method comprises administering by convection-enhanced delivery (CED) for a period of up to 96 hours a therapeutically effective amount of a mutagenized IL13 linked to a cytotoxin (cmIL13).

### CROSS-REFERENCE TO RELATED APPLICATION

This application claims the benefit of U.S. Provisional Patent Application Ser. No. 63/277,866 filed on Nov. 10, 2021, incorporated herein by reference.

### GOVERNMENT CONTRACT

This disclosure was made with Government support under Government Contract No. R43 CA275560 awarded by the National Institutes of Health. The United States Government has certain rights in the invention.

### FIELD

The present disclosure relates to cancer therapy.

### BACKGROUND

Prognosis for malignant gliomas remains poor due to the lack of effective therapeutic treatments. The limited effectiveness of the current therapeutic modalities for certain cancers highlights the necessity for the development of effective and novel treatments for patients suffering from these diseases.

### SUMMARY

Disclosed herein are methods of treating a subject having a malignant glioma comprising: administering by convection-enhanced delivery (CED) for a period of up to 96 hours a therapeutically effective amount of a mutagenized IL13 (mIL13) linked to a cytotoxin (cmIL13); wherein the malignant glioma expresses an interleukin 13 receptor α 2 (IL13Rα2).

Also disclosed herein is a mutagenized IL13 linked to a cytotoxin (cmIL13) for use in a method of treating a subject having a malignant glioma expressing IL13Rα2, wherein the method comprises administering by convection-enhanced delivery (CED) for a period of up to 96 hours a therapeutically effective amount of a cmIL13.

### BRIEF DESCRIPTION OF THE DRAWINGS

FIG. 1A. RNA-Seq of diffuse midline glioma (DMG) and adult glioblastoma (GBM) cell models.

FIG. 1B. Immunoblotting of DMG and GBM cell models.

FIG. 2A. Cell proliferation and dose-response curve of DMG cell lines, shown as a percent response as a function of cmIL13 dosage.

FIG. 2B. Cell viability and dose-response curve of DMG cells lines, shown as a percent response as a function of cmIL13 dosage.

FIG. 2C. Graph of the inverse relationship between IL13Rα2 expression of DMG cells and sensitivity towards cmIL13.

FIG. 2D. Immunoblotting of SU-DIPG XIII-P, SF8628, and PED17 cell lines after 8, 24, 48, and 72 hours of wild type IL13 (10 ng/mL) exposure.

FIG. 2E. Immunoblotting of SU-DIPG XIII-P, SF8628, and PED17 cell lines after 8, 24, 48, and 72 hours of cmIL13 (cell line-specific IC₅₀) exposure.

FIG. 2F. Immunofluorescence staining of SF8628 cells following 72 hours of treatment with cmIL13 at IC₅₀.

FIG. 3. Cell viability and dose-response curve of adult GBM cell lines, shown as a percent response as a function of cmIL13 dosage.

FIG. 4. cmIL13 IC₅₀ values for DMG and GBM cell lines as a function of IL-13Rα2 expression.

FIG. 5A. A schematic representation of tumor cell injection and CED workflow.

FIG. 5B. Biolumiscence (BLI) signals as a function of days post injection of 1 μg of cmIL13 for GBM6-bearing animals.

FIG. 5C. Prolonged survival of GBM6-bearing animals as shown by percent survival as a function of days post-injection of 1 μg of cmIL13.

FIG. 5D. BLI signals as a function of days post-injection of 1 μg of cmIL13 for PED17 xenografts.

FIG. 5E. Prolonged survival of PED17 xenografts as shown by percent survival as a function of days post-injection of 1 μg of cmIL13.

FIG. 5F. BLI signals as a function of days post-injection of 1 μg of cmIL13 for SU-DIPG-XIII-P animals.

FIG. 5G. Prolonged survival of SU-DIPG-XIII-P animals as shown by percent survival as a function of days post-injection of 1 μg of cmIL13.

FIG. 6A. Immunohistochemistry of HGG-bearing mouse brains harvested on day 55 (control) and day 86 (experimental) following CED of vehicle solution or 1 μg of cmIL13, respectively.

FIG. 6B. IHC analysis of IL-13Rα2 levels, apoptosis induction, and cellular proliferation in mice treated with cmIL13 compared to control.

FIG. 6C. The density of cells containing Ki-67, cleaved caspase 3, and NeuN for controls and mice treated with 1 μg of cmIL13.

FIG. 7A. Immunofluorescence (IF) of SF8628 cells after 8, 24, 48, and 72 hours of cmIL13 exposure at IC₅₀. The cmIL13 is shown in red, the IL-13Rα2 is shown in green, and DAPI is shown in blue.

FIG. 7B. IF of SF8628 cells after 8, 24, 48, and 72 hours of cmIL13 exposure at IC₅₀. Cleaved caspase 3 is shown in green, Ki-67 is shown in red, and DAPI is shown in blue.

### DETAILED DESCRIPTION

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, the preferred materials and methods are described herein.

For purposes of the following detailed description, it is to be understood that the disclosure may assume various alternative variations and step sequences, except where expressly specified to the contrary. Moreover, other than in any operating examples, or where otherwise indicated, all numbers such as those expressing values, amounts, percentages, ranges, subranges, and fractions may be read as if prefaced by the word “about.” even if the term does not expressly appear. Accordingly, unless indicated to the contrary, the numerical parameters set forth in the following specification and attached claims are approximations that may vary depending upon the desired results to be obtained by the present disclosure. At the very least, and not as an attempt to limit the application of the doctrine of equivalents to the scope of the claims, each numerical parameter should at least be construed in light of the number of significant digits and by applying ordinary rounding techniques. Where a closed or open-ended numerical range is described herein, all numbers, values, amounts, percentages, subranges, and fractions within or encompassed by the numerical range are to be considered as being specifically included in and belonging to the original disclosure of this application as if these numbers, values, amounts, percentages, subranges, and fractions had been explicitly written out in their entirety.

Notwithstanding that the numerical ranges and parameters that set forth the broad scope of the disclosure are approximations, the numerical values set forth in the specific examples are reported as precisely as possible. Any numerical value, however, inherently contains certain errors necessarily resulting from the standard variation found in their respective testing measurements.

As used herein, unless indicated otherwise, a plural term can encompass its singular counterpart and vice versa. For example, although reference is made herein to “a” mutagenized IL13, or “a” cytotoxin, a combination (i.e., a plurality) of these components can be used. In addition, in this application, the use of “or” means “and/or” unless specifically stated otherwise, even though “and/or” may be explicitly used in certain circumstances.

As used herein, “including,” “containing” and like terms are understood in the context of this application to be synonymous with “comprising” and are therefore open-ended and do not exclude the presence of additional undescribed and/or unrecited elements, materials, ingredients, and/or method steps.

As used herein, “consisting of” is understood in the context of this application to exclude the presence of any unspecified element, ingredient, and/or method step.

As used herein, “consisting essentially of” is understood in the context of this application to include the specified elements, materials, ingredients, and/or method steps “and those that do not materially affect the basic and novel characteristic(s)” of what is being described.

As used herein, “patient,” “subject,” “individual,” and the like are used interchangeably and refer to any animal or cell thereof, whether in vitro or in situ, including mammals, including a human, a canine, a feline, a bovine, an equine, a porcine, a primate, and/or a rodent.

As used herein, the term “interleukin-13” or “IL13” means any native or wild type IL13 from any vertebrate source, including mammals such as primates and rodents, unless indicated to the contrary, and includes unprocessed IL13 and any form of IL13 that results from processing in a cell and any naturally occurring variants of IL13, such as splice or allelic variants. The amino acid sequence of an exemplary human IL13 is shown in SEQ ID NO:1. An amino acid sequence of a second exemplary human IL13 is shown in SEQ ID NO:2.

A “mutation” in a polypeptide is meant to encompass proteins having any amino acid substitutions, deletions (e.g., a truncated version of the protein, such as a peptide), insertions, and/or modifications, such as by glycosylation, phosphorylation, acetylation, myristoylation, prenylation, palmitoylation, amidation, and the like. In an example, a “mutated IL13” or “mutagenized IL13” refers to an IL13 in which one or more of the amino acids differ from the corresponding amino acids in the native form of IL13. Mutated IL13 and/or mutagenized IL13 may be derived from the native form of IL13 found in humans, non-human primates, rats, murine, porcine, bovine, canine, and the like. A mutated IL13 and/or mutagenized IL13 may be referred to herein as “mIL13.”

As used herein, the term “IL-13 receptor” or “IL13R” refers to a receptor that binds IL-13.

As used herein, the term “IL-13 receptor α 2” or “IL13Rα2” refers to the monomeric IL13 receptor that is expressed on the surface of specific cell subsets and that binds IL13.

As used herein, the term “cmIL13” or “cmIL-13” refers to a mutagenized IL13 linked to a cytotoxin.

As used herein, “treat,” “treatment,” or “treating” means a therapeutic measure provided to a patient or subject with the intention of preventing the development or altering the pathology or symptoms experienced by the patient or subject, such as, those resulting from a disorder. A “treatment” administered to a patient or subject may achieve any clinically or quantitatively measurable reduction in the condition for which the patient or subject is being treated up to and including complete elimination.

As used herein, a “therapeutically effective amount” is defined as an amount that, when administered to a patient in order to treat a disease (e.g., cancer) is sufficient to treat the disease. For example, a therapeutically effective amount of a compound for treatment of cancer may be, e.g., the amount sufficient to decrease the volume of a malignant tumor or to prolong survival of the patient.

As used herein, a “cytotoxin” is defined as a substance, such as a toxin or antibody, that inhibits the function of cells, causes cell destruction, or both.

As used herein, the term “affinity” is defined as the strength of the total of non-covalent interactions between a single binding site of a receptor and a ligand. The affinity of a receptor for a ligand can be expressed by the dissociation constant (K_{D}), which is the ratio of dissociation and association rate constants K_{off} and Kₒₙ, respectively. Affinity can be measured by methods known to those of skill in the art.

“Increased binding” refers to binding levels of a mIL13 which are at least 10% or more, such as 20%, 30%, 40%, 50%, 60%, 70%, 80%, 90% higher or more, or 1-fold, 2-fold, 5-fold, 10-fold, 20-fold, 100-fold, 1000-fold higher or more, and any and whole or partial increments therebetween, than a wild type IL13.

“Decreased binding” refers to binding levels of a mIL13 which are at least 10% or more, such as 20%, 30%, 40%, 50%, 60%, 70%, 80%, 90% lower or less, or 1-fold, 2-fold, 5-fold, 10-fold, 20-fold, 100-fold, 1000-fold lower or less, and any and whole or partial increments therebetween, than a wild type IL13.

As used herein, the term “link” is defined as a chemical attachment (covalent or non-covalent) of one gene to a second gene, for example, by conjugation or fusion. As used herein, the term “linking” is defined as chemically attaching (covalently or non-covalently) one gene to a second gene. As used herein, the term “conjugation” is defined as the linking of one gene to a second gene post-translation. As used herein, the term “fusion” is defined as the linking of one gene to a second gene pre-translation.

As used herein, the terms “control” or “reference” or “comparator control” are defined as a subject that is not administered a cmIL13 by convection enhanced delivery, such that the control or reference standard may serve as a comparator against which an experimental sample can be compared.

As used herein, the term “determining the level of marker (or biomarker) expression” is meant as an assessment of the degree of expression or presence of a marker in a sample at the nucleic acid or protein level, using technology available to the skilled artisan to detect a sufficient portion of any marker expression product, such as “determining the level of IL13Rα2.”

As used herein, the “level” of one or more marker (or biomarker) means the absolute or relative amount or concentration of the marker (or biomarker) in the sample.

“Measuring” or “measurement,” or alternatively “detecting” or “detection,” means assessing the presence, absence, quantity, or amount (which can be an effective amount) of either a given substance within a clinical or subject-derived sample, including the derivation of qualitative or quantitative concentration levels of such substance, or otherwise evaluating the values or categorization of a subject's clinical parameters.

“Sample” or “biological sample” as used herein means a biological material isolated from an individual, such as a liquid or solid biological sample collected via a biopsy (i.e., a “liquid biopsy” or a “solid biopsy”). A liquid biopsy may comprise, for example, blood, plasma, saliva, urine, cerebral spinal fluid, and/or other body fluid. The liquid biopsy may contain extracellular vesicles and/or cell-free genetic material. A solid biopsy may comprise, for example, an organ and/or a tissue, such as a tumor. The biological sample may contain any biological material suitable for detecting the desired biomarkers and may comprise cellular and/or non-cellular material obtained from the individual.

The term “cancer” as used herein is defined as a disease characterized by the abnormal growth of aberrant cells. Cancer cells can spread locally or through the bloodstream and lymphatic system to other parts of the body. Examples of various cancers include those brain cancers in which tissue cells express IL13Rα2, including but not limited to gliomas and the like.

The term “glioma” is defined as a tumor that originates in glial cells of the brain or spinal cord.

The present disclosure is directed to methods of treating a subject having a malignant glioma comprising, consisting essentially of, or consisting of administering by convection-enhanced delivery (CED) for a period of up to 96 hours a therapeutically effective amount of a mutagenized IL13 linked to a cytotoxin (cmIL13); wherein the malignant glioma expresses an interleukin 13 receptor α 2 (IL13Rα2).

The present disclosure is also directed to a mutagenized IL13 linked to a cytotoxin (cmIL13) for use in a method of treating a subject having a malignant glioma expressing IL13Rα2, wherein the method comprises administering by convection-enhanced delivery (CED) for a period of up to 96 hours a therapeutically effective amount of a cmIL13.

The method comprises, consists essentially of, or consists of administration of a mutagenized IL13 (mIL13). The mIL13 may be linked to a cytotoxin (cmIL13).

The mIL13 may be engineered to have increased affinity for IL13Rα2 compared to native human IL13 and/or decreased affinity for interleukin 13 receptor α 1 (IL13Rα1) compared to native human IL13. For example, the mIL13 may have a reduced affinity to the IL13Rα1 compared to a wild-type polypeptide, for example, a 2-fold decrease, a 3-fold decrease, a 5-fold decrease, a 10-fold decrease, a 100-fold decrease, or more of kinetic K_{D} while at least retaining binding or activation of the IL13Rα2. In some instances, the affinity of the mIL13 to the IL13Rα1 is completely impaired such that no binding is detectable by K_{D}, while retaining binding or activation of the IL13Rα2. In some instances, the affinity of the mIL13 for the IL13Rα2 is at least retained, or may in some instances be increased, compared to a wild-type IL13, for example, a 2-fold increase, a 3-fold increase, a 5-fold increase, a 10-fold increase, a 100-fold increase, a 1000-fold increase, or more of kinetic K_{D}.

In examples, the mIL13 may be produced by cDNA mutagenesis, DNA synthesis, peptide/protein synthesis, or any method known to those of skill in the art.

The mIL13 may be a full-length IL13 molecule, such as a human full-length IL13 molecule. In examples, the mIL13 may comprise amino acid changes relative to wild type IL13 at a position corresponding to residue 13 of the human IL13 (SEQ ID NO:1), at a position corresponding to residue 66 of SEQ ID NO: 1, at a position corresponding to residue 69 of SEQ ID NO: 1, and/or at a position corresponding to residue 105 of SEQ ID NO: 1. The mIL13 may comprise a substitution to the glutamic acid at a position 13 of the human IL13 (SEQ ID NO:1). For example, lysine may be substituted for the glutamic acid at position 13. The mIL13 may comprise a substitution to the arginine at position 66 of the human IL13 (SEQ ID NO:1). For example, aspartic acid may be substituted for the arginine at position 66. The mIL13 may comprise a substitution to the serine at position 69 of the human IL13 (SEQ ID NO:1). Aspartic acid may be substituted for the serine at position 69. The mIL13 may comprise a substitution of the lysine at position 105 the human IL13 (SEQ ID NO:1). Arginine may be substituted for the lysine at position 105. The mIL13 may comprise changes at positions E13, R66, S69, and/or K105. The mIL13 may be designated IL13.E13K.R66D.S69D.K105R. In an example, the mIL13 may comprise the amino acid sequence set forth in SEQ ID NO:3. In other examples, the mIL13 may comprise any of SEQ ID NOS: 4 to 22, or may comprise a sequence sharing at least 50, 60, 70, 80, 85, 90, 95, or 99% homology with any of SEQ ID NOS: 1 to 24, or may comprise a sequence that differs by no more than 1, 2, 3, 4, 5, 6, 7, 8, 9, 10, 15, or 20 residues from any of SEQ ID NOS: 1 to 24. In examples, U.S. Publ. Ser. No. 15/597,823, filed on May 17, 2017, and entitled “Therapeutic IL13 Polypeptides,” col. 2, ll. 16-47; col. 2 l. 56 to col. 3, In. 3, incorporated herein by reference, discloses a wild type IL13 (disclosed herein as SEQ ID NO:2) and IL13Rα2-specific mutations provided herein as SEQ ID NOS: 3 to 24. The term “homology” and “homologous” refers to the subunit sequence identity between two molecules, such as two protein or peptide molecules. When a subunit position in both of the molecules is occupied by the same subunit, then the molecules are homologous at that position. The homology between two sequences is a direct function of the number of matching or homologous positions. For example, if half of the positions in two sequences are homologous, then the two sequences are 50% homologous and if 70% of the positions (i.e., 7 of 10) are matched or homologous, then the two sequences are 70% homologous. Homologs of the present disclosure can be the result of natural allelic variation, including natural mutation. Homologs of the present disclosure can be the result of natural allelic variation, including natural mutation. Homologs of the present disclosure can also be produced using techniques known in the art, including direct modifications to the protein, using, for example, recombinant DNA techniques to effect random or targeting mutagenesis.

The mIL13 may be complexed with an extracellular vesicle (EV) to form an extracellular vesicle complex (EV complex). The extracellular vesicle may be obtained from a particular type of biological sample (urine, serum, plasma, cerebrospinal fluid, an organ, a tissue, and the like) and/or may be derived from a particular type of cell, such as glioma stem cells. In examples, the glioma stem cells may be mesenchymal glioma stem cells or proneural glioma stem cells. The extracellular vesicle may comprise an exosome, such as a tumor-associated exosome. The extracellular vesicles may be purified or concentrated from a biological sample using differential centrifugation, ultracentrifugation, and/or other methods known to those of skill in the art.

Subpopulations of extracellular vesicles may be isolated by using a biological marker. The biological marker may be a receptor, such as a tumor-associated receptor. The tumor-associated receptor may be IL13Rα2. Each exosome may, for example, express 1, 2, 5, 10, 15, 20, 25, 50, 100, 250, 500, 1000 or more biological markers.

A cytotoxin may be linked to one of the mIL13 disclosed herein to form a cmIL13. For example, the linking may occur by EDC chemistry or purified gel filtration.

The cytotoxin may comprise, consist essentially of, or consist of a bacterial-derived toxin. The bacterial-derived toxin may comprise, for example, Pseudomonas exotoxin A.

A therapeutically effective amount of cmIL13 may be administered by convection-enhanced delivery (CED). CED comprises directly inserting at least one catheter into, for example, the interstitial spaces of the brain, into a resection cavity of the brain, or into an intact brain tumor. One skilled in the art will be able to select an appropriate catheter for delivery of cmIL13 via CED. As used herein, “convection enhanced delivery” or “CED” is defined as a method of low-flow positive pressure infusion that administers therapeutic agents directly to the structure to be treated, e.g., a malignant glioma tumor or a resection cavity. The cmIL13 may be administered by convective infusion at a specified flow rate controlled by an external syringe pump. The method comprises positioning the tip of a catheter within the area to be treated. An external pump may be connected to the catheter, which supplies a composition comprising a therapeutically effective dose of a therapeutic agent, e.g., cmIL13, while maintaining a positive pressure gradient through delivery. Administration of cmIL13 may or may not occur with co-infusion of an imaging component to serve as a surrogate marker for drug delivery, e.g., gadolinium or gadolinium-DTPA.

Administration of the cmIL13 may occur over a period of at least 4 hours. In examples, administration of the cmIL13 may occur over a period of 4 hours to 96 hours.

The cmIL13 may be administered at a dose of at least 0.03 μg/mL to no greater than 1 μg/mL. The administration of the cmIL13 may be administered at a flow rate of up to 1 mL/hour.

The malignant glioma treated by the method disclosed herein may comprise, consist essentially of, or consist of a high-grade glioma. Examples of malignant gliomas that may be treated with the method disclosed herein include but are not limited to an adult glioblastoma, a pediatric glioblastoma, an anaplastic astrocytoma, such as a diffuse midline glioma, an anaplastic oligodendroglioma, an anaplastic oligoastrocytoma, an anaplastic ependymoma, and/or an anaplastic ganglioma.

The malignant glioma treated with the disclosed method may express an IL13 specific receptor. The IL13 specific receptor may comprise interleukin 13 receptor α 2 (IL13Rα2). Prior to administration of the cmIL13, expression of IL13Rα2 may be detected in a sample of the malignant glioma. In examples, the sample of the malignant glioma may be purified prior to detection of expression of IL13Rα2. Detecting expression of IL13Rα2 may be detected by mass spectrometry, analytical assay, immunostaining, and/or sequencing. In examples, the analytical assay may comprise an enzyme-linked immunosorbent assay (ELISA). In other examples, the immunostaining may comprise immunohistochemistry (IHC) and/or fluorescent cytochemistry. In other examples, the sequencing comprises whole genome sequencing, exome sequencing, proteomic sequencing, and/or RNA sequencing. Expression of the IL13Rα2 by the malignant glioma is indicative that the malignant glioma will be responsive to treatment by the cmIL13.

It has been surprisingly discovered that the method of treating a malignant glioma described herein increases survival of a subject having a malignant glioma relative to a control subject having a malignant glioma that is not treated by the method described herein. The increase in survival may be determined, for example, by Kaplan-Meier survival curve analyses. Survival curve analysis includes, but is not limited to, comparing the survival probability between cmIL13 and control treated subjects where survival probability (St) is defined as [Number of living subjects at start−Number of subjects died]/Number of living subjects at start. The resultant data is plotted on a graph whereby the survival probability is on the Y axis and the time past entry in the study is on the X axis. Statistical difference between the at least two different graphed data sets can be determined by at least either log rank test, whereby the chi-square for each even time for each group is calculated and sums the results, or by hazard ratio whereby the calculation of chi-square for each event time and sum the results, giving the final observed and expected results of the full curve. Statistical significance is defined as p being equal to or less than 0.05.

It has also been surprisingly discovered that the method of treating a malignant glioma described herein results in no detectible changes in clinically relevant biomarkers within healthy cells and/or healthy tissues (e.g., non-glioma cells and non-glioma tissues). Examples of clinically relevant biomarkers include but are not limited to NeuN and CD68+ proteins.

Finally, it was surprisingly discovered that the method of treating a malignant glioma disclosed herein results in a reduction in the volume of the malignant glioma. The reduction in volume of the glioma can be determined by statistical analyses known to those skilled in the art, for example, by a one-way or a two-way Analysis of Variants (ANOVA) test or a two-tailed Student's t-test, wherein statistical significance may be determined by a p<0.05. Therefore, the method of treating a malignant glioma described herein can effectively treat malignant gliomas while not harming healthy cells and tissue.

### Aspects

In the following, some non-limiting aspects of the present disclosure are summarized:

Aspect 1. A method of treating a subject having a malignant glioma comprising:
administering by convection-enhanced delivery (CED) for a period of up to 96 hours a therapeutically effective amount of a mutagenized IL13 (mIL13) linked to a cytotoxin (cmIL13);
wherein the malignant glioma expresses an interleukin 13 receptor α 2 (IL13Rα2).

Aspect 2. The method of Aspect 1 wherein the cmIL13 is administered for a period of 4 hours to 96 hours.

Aspect 3. The method of Aspect 1 or Aspect 2, wherein the cmIL13 is administered at a dose of 0.03 μg/mL to 1 μg/mL.

Aspect 4. The method of any of the preceding Aspects, wherein administration of the cmIL13 is administered at a flow rate of up to 1 mL/hour.

Aspect 5. The method of any of the preceding Aspects, wherein the cmIL13 is engineered to have increased affinity for IL13Rα2 compared to native human IL13 and/or decreased affinity for interleukin 13 receptor α 1 (IL13Rα1) compared to native human IL13.

Aspect 6. The method of any of the preceding Aspects, wherein the cmIL13 comprises amino acid changes relative to wild type IL13 at positions E13, R66, S69, and/or K105.

Aspect 7. The method of any of the preceding Aspects, wherein the cmIL13 comprises one or more amino acid substituents E13K.R66D.S69D.K105R.

Aspect 8. The method of any of the preceding Aspects, wherein the cmIL13 comprises an amino acid sequence set forth in one of SEQ ID NOS: 3 to 24 or a homologue thereof or a homologue of one of SEQ ID NO:1 or SEQ ID NO:2.

Aspect 9. The method of any of the preceding Aspects, wherein the cmIL13 differs by no more than 20 residues from SEQ ID NO: 1 or SEQ ID NO:2.

Aspect 10. The method of any of the preceding Aspects, wherein the cytotoxin comprises a bacterial-derived toxin.

Aspect 11. The method of Aspect 10, wherein the bacterial-derived toxin comprises Pseudomonas exotoxin A.

Aspect 12. The method of any of the preceding Aspects, wherein the malignant glioma comprises a high-grade glioma.

Aspect 13. The method of any of the preceding Aspects, wherein the malignant glioma comprises an adult glioblastoma, a pediatric glioblastoma, an anaplastic astrocytoma, an anaplastic oligodendrogliomas, an anaplastic oligoastrocytomas, an anaplastic ependymomas, and/or an anaplastic gangliomas.

Aspect 14. The method of Aspect 13, wherein the anaplastic astrocytoma comprises a diffuse midline glioma.

Aspect 15. The method of any of the preceding Aspects, further comprising co-infusion of an imaging component.

Aspect 16. The method of Aspect 15, wherein the imaging component comprises gadolinium, gadolinium-DTPA, or a combination thereof.

Aspect 17. The method of any of the preceding Aspects, further comprising detecting expression of IL13Rα2 in a sample, wherein expression of the IL13Rα2 is indicative of the malignant glioma being responsive to treatment by cmIL13.

Aspect 18. The method of Aspect 17, wherein the sample comprises:
a liquid biopsy of blood, plasma, saliva, urine, and/or cerebral spinal fluid; and/or
a solid biopsy of an organ and/or a tissue.

Aspect 19. The method of Aspect 18, wherein the tissue comprises a tumor.

Aspect 20. The method of Aspect 19, wherein the tumor comprises the malignant glioma.

Aspect 21. The method of any of Aspects 18 to 20, wherein the liquid biopsy comprises an extracellular vesicle and/or cell-free genetic material.

Aspect 22. The method of any of Aspects 18 to 21, further comprising purifying the sample.

Aspect 23. The method of any of Aspects 18 to 22, wherein detecting expression of IL13Rα2 is done by mass spectrometry, analytical assay, immunostaining, and/or sequencing.

Aspect 24. The method of Aspect 23, wherein:
(a) the analytical assay comprises an enzyme-linked immunosorbent assay (ELISA);
(b) the immunostaining comprises immunohistochemistry (IHC) and/or fluorescent cytochemistry; and/or
(c) the sequencing comprises whole genome sequencing, whole exome sequencing, RNA sequencing, and/or proteomic sequencing.

Aspect 25. The method of any of the preceding Aspects, wherein the administering results in an increase in survival of the subject relative to a control subject having a malignant glioma that is not administered a therapeutically effective amount of the cmIL13.

Aspect 26. The method of Aspect 25, wherein the increase in survival is determined by Kaplan-Meier survival curve analyses.

Aspect 27. The method of any of the previous Aspects, wherein the method results in no detectible changes in clinically relevant biomarkers within non-glioma cells and/or non-glioma tissue.

Aspect 28. The method of Aspect 27, wherein the biomarkers comprise NeuN and CD68+ proteins.

Aspect 29. The method of any of the preceding Aspects, wherein the administration of the cmIL13 to cells expressing IL13Rα2 results in:
(i) a reduction in volume of the malignant glioma;
(ii) an increase in detectable cleaved caspase 3, as measured by IHC or fluorescent cytochemistry; and/or
(iii) a reduction in the level of Ki-67-positive cells by an amount of at least 10%, as measured by IHC or fluorescent cytochemistry.

Aspect 30. The method of Aspect 29, wherein the reduction in volume of the malignant glioma is determined by an Analysis of Variants (ANOVA) test, wherein statistical significance is determined by a p value of <0.05.

Aspect 31. The method of Aspect 29, wherein the reduction in volume of the malignant glioma is determined by a two-tailed Student's t-test, wherein statistical significance is determined by a p value<0.05.

Aspect 32. The method of any of the preceding Aspects, wherein the mutagenized IL-13 is characterized by at least one amino acid substitution as compared to SEQ ID NO: 1 or SEQ ID NO:2.

Aspect 33. A mutagenized IL13 linked to a cytotoxin (cmIL13) for use in a method of treating a subject having a malignant glioma expressing interleukin 13 receptor α 2 (IL13Rα2), wherein the method comprises administering by convection-enhanced delivery (CED) for a period of up to 96 hours a therapeutically effective amount of a cmIL13; and/or the method of treatment is optionally further characterized by any of the features recited in the preceding Aspects.

Illustrating the disclosure are the following examples which, however, are not meant to be considered as limiting the disclosure to their details. Unless otherwise indicated, all parts and percentages in the following examples and throughout the specification are by weight.

### Examples

### Study 1

### Materials and Methods:

### Materials

cmIL13 (IL13.E13K-PE4E) was obtained from Targepeutics, Inc. (Hershey, PA). cmIL13 was dissolved in Phosphate-Buffered Saline (PBS) and stored as 2.6 mg/mL stock at −80° C. Human IL13 recombinant protein (Cat No. A2525) was obtained from Invitrogen (Thermo Fisher Scientific). IL13 was dissolved in double-distilled water (ddH2O) per manufacturer's protocol and stored as 5 μg/mL stock at −80° C.

### Cell Lines and Culture

Informed consent and Institutional Review Board approval were obtained for all patient-derived cell lines. Details regarding cell lines are shown in Table 1.

TABLE 1
Details regarding the cell lines.

| | Age/ | | | | Date of Tissue | Culture |
|---|---|---|---|---|---|---|
| Cell line ID | Sex | Institution | Molecular Status | Tests | Collection | Media |
| GBM6 | 65/M | Mayo Clinic | GBM, IDH WT | WES, Sanger, STR | 2000 | FBS or Stem |
| GBM8 | 75/F | Mayo Clinic | GBM, IDH WT | WES, Sanger, STR | 2000 | FBS or Stem |
| GBM10 | 40/M | Mayo Clinic | GBM, IDH WT | WES, Sanger, STR | 2001 | FBS or Stem |
| GBM12 | 69/M | Mayo Clinic | GBM, IDH WT | WES, Sanger, STR | 2001 | Stem |
| GBM14 | 58/M | Mayo Clinic | GBM, IDH WT | WES, Sanger, STR | 2001 | FBS or Stem |
| GBM26 | 49/M | Mayo Clinic | GBM, IDH WT | WES, Sanger, STR | 2002 | FBS |
| GBM39 | 51/M | Mayo Clinic | GBM, IDH WT | WES, Sanger, STR | 2003 | FBS or Stem |
| GBM43 | 69/M | Mayo Clinic | GBM, IDH WT | WES, Sanger, STR | 2003 | FBS or Stem |
| GBM59 | 83/F | Mayo Clinic | GBM, IDH WT | WES, Sanger, STR | 2004 | FBS |
| GBM64 | 63/M | Mayo Clinic | GBM, IDH WT | WES, Sanger, STR | 2006 | FBS or Stem |
| GBM108 | 62/M | Mayo Clinic | GBM, IDH WT | WES, Sanger, STR | 2009 | FBS or Stem |
| GBM118 | 56/F | Mayo Clinic | GBM, IDH WT | WES, Sanger, STR | 2009 | FBS |
| GBM123 | 62/F | Mayo Clinic | GBM, IDH WT | WES, Sanger, STR | 2009 | FBS or Stem |
| GBM134 | 42/F | Mayo Clinic | GBM, IDH WT | WES, Sanger, STR | 2009 | FBS |
| GBM161 | 78/M | Mayo Clinic | Glioscarcoma, IDH WT | WES, Sanger, STR | 2012 | FBS |
| PED17 | 12/F | Mayo Clinic | DMG, H3.3 K27M | Sanger, STR | 2015 | MHM |
| SU-DIPG XIII-P | 6/F | Stanford University | DMG, H3.3 K27M | Sanger, STR | Received 2016 | TSM |
| SU-DIPG XVII | 8/M | Stanford University | DMG, H3.3 K27M | Sanger, STR | Received 2016 | MHM |
| SF8628 | 3/F | Millipore | DMG, H3.3 K27M | Sanger, STR | Purchased 2019 | dBT |
| SF8628-B23 | — | Hormel Institute | DMG, H3.3 K27M KO | Sanger, STR | Created 2019 | dBT |

Abbreviations:
GBM = glioblastoma,
IDH = isocitrate dehydrogenase,
DMG = diffuse midline glioma,
WES = whole exome sequencing,
STR = short tandem repeat,
WT = wildtype.

Early passage HGG lines were used, and all cell lines were validated by short tandem repeat DNA fingerprinting annually and tested for Mycoplasma contamination every 3 months. Cell lines with the H3K27M mutation were validated for K27M-mutant histone expression using Western blot and Sanger sequencing every 3 months. All patient-derived tumor cell lines were maintained in cell-line appropriate medium, the details of which are provided in Table 2.

TABLE 2
Media composition for culture of patient-derived cell lines.

| Cell Lines | Media Name | Media Contents |
|---|---|---|
| GBM6, GBM8, GBM10, GBM14, | FBS-containing | DMEM (Thermo Fisher), 10% PBS (Atlanta Biolocials), |
| GBM26, GBM39, GM43, GBM59, | medium | and 1% penicillin/streptomycin (Thermo Fisher). |
| GBM64, GBM108, GBM118, | (FBS) | |
| GBM123, GBM134, GBM161 | | |
| GBM6, GBM8, GBM10, GBM12, | Stem cell | KnockOut DMEM/F12 (Thermo Fisher), StemPro NSC SFM |
| GBM14, GBM39, GM43, GBM64, | medium | supplement (Thermo Fisher), 20 ng/mL FGF basic recombinant |
| GBM108, GBM123 | (Stem) | human (Thermo Fisher), 20 ng/mL EGF recombinant |
| | | human(Thermo Fisher), 200 mM L-glutamine (Corning), and 1% |
| | | penicillin/streptomycin (Thermo Fisher). |
| PED17, SU-DIPG XVII | Serum-free | DMEM/F12 (HyClone), 25 mM glucose (Sigma-Akirich), 8.9 |
| | complete | mM sodium bicarbonate (Life Technologies), 2 mM glutamine |
| | medium | (Life Technologies), 4 mM HEPES (Life Technologies), 1% |
| | (MHM) | penicillin/streptomycin (Life Technologies), N2 supplement |
| | | (Life Technologies), 4 μg/mL heparin (Sigma Aldrich), |
| | | 20 ng/mL human EGF (PeproTech US), 20 ng/mL human b-FGF |
| | | (PeproTech US), and 20 ng/mL human PDGP AA with 20 |
| | | ng/mL human PDGF BB (Shenandoah Biotechnology). |
| SU-DIPG XIII | Tumor stem | Neurobasal(-A) (Life Technologies), DMEM/F12 (Life |
| | cell medium | Technologies) B27(-A) (Life Technologies), 4 μg/mL heparin |
| | (TSM) | (Sigma Aldrich), 20 ng/mL human EGF (PeproTech US), 20 |
| | | ng/mL human b-FGF (PeproTech US), 20 ng/mL haman PDGF |
| | | AA with 20 ng/mL human PDGF BB (Shenandoah |
| | | Biotechnology), and 1% penicillin/streptomycin (Lite |
| | | Technologies). |
| SP8628, SF8628-B23 | dBT medium | DMEM/F12 (HyClone), 10% FBS, and 1% |
| | | penicillin/streptomycin (Life Technologies). |

Cells cultured as neurospheres were passaged every 1-2 weeks. Cells cultured as adherent monolayers were passaged 1-2 times per week.

### RNA Sequencing and Data Analysis

Total RNA was extracted from whole-cell lysates using the RNeasy Plus micro kit (Cat #74034; QIAGEN, Germantown, MD, USA) according to the manufacturer's instructions. For the purpose of screening a large library of cell lines, RNA-Seq studies were performed as single replicates. RNA library preparation and sequencing were performed by Novogene (Beijing, China). The NEBNext Ultra™ RNA Library Prep Kit for Illumina sequencers (New England Biolabs, Ipswich, MA, USA) was used for library preparation, and cDNA libraries were subsequently size-selected using AMPure XP magnetic beads (Beckman Coulter, Pasadena, CA, USA). Samples were sequenced on an NovaSeq 6000 sequencer (Illumina, San Diego, CA, USA) using either single- or paired-end sequencing, depending on the timeframe of sample availability and the sequencing technology available. Paired-end sequencing data on adult GBM cell lines was obtained from cBioPortal, a free web-based tool that contains RNA-Seq data on Mayo Clinic's brain tumor patient-derived xenografts. Generated FASTQ files underwent quality assessment using FASTQC. Trimmed reads were mapped to hg38 using STARv2.7.3a, and annotated gene counts were obtained using the -quantMode geneCounts function. Transcripts (TPM) or reads per kilo base per million (RPKM) reads values were calculated using RSEM in a manner concordant with the single- or paired-end status of the library.

### Immunoblotting

Patient-derived tumor cells for immunoblotting were lysed in Triton X-100 lysis buffer containing protease inhibitors and sonicated. Collected protein lysates were stored at −20° C. Protein concentrations were determined using the Pierce BCA Protein Assay Kit (Cat #23227; Thermo Fisher Scientific). Fifteen μg of total protein was size fractioned by 12.5% SDS-PAGE. Electrophoresis-separated proteins were electrically transferred to a polyvinylidene difluoride (PVDF) membrane, washed in PBST buffer, and blocked in 2% fat-free milk for 1 h at room temperature, then incubated with primary antibodies at 4° C. overnight. Following primary antibody blotting, specific signal was detected with species-appropriate peroxidase-conjugated secondary antibody (Thermo Fisher Scientific) using SuperSignal West Pico PLUS Chemiluminescent Substrate (Cat #34580; Thermo Fisher Scientific) and imaged using an Azure 600 Western blot imaging system (Azure Biosystems, Dublin, CA, USA). Details regarding antibodies used for Western blots can be found in Table 3.

TABLE 3
Antibodies used for immunoblotting, immunofluorescence,
and immunohistochemistry.

| | | | Dilution | Dilution | Dilution |
|---|---|---|---|---|---|
| Antibodies | Source | Identifier | for WB | for IF | for IHC |
| Cleaved PARP | Cell Signaling | 5625S | 1:1000 | | |
| Cleaved Caspase 3 | Cell Signaling | 9664S | 1:1000 | 1:500 | 1:200 |
| IL-13Rα1 | Abcam | Ab79277 | 1:1000 | | |
| IL-13Rα2 | Cell Signaling | 85677 | 1:1000 | 1:200 | |
| H3K27M | Abcam | 190631 | 1:3000 | | 1:500 |
| H3K27me3 | Cell Signaling | 9733S | 1:1000 | | 1:100 |
| Histone H3 | Cell Signaling | 3638S | 1:1000 | | |
| α-Tubulin | Sigma | T9026 | 1:20,000 | | |
| Vinculin | Cell Signaling | 13901S | 1:1000 | | |
| Pseudomonas Exotoxin A | Sigma | P2318 | | 1:500 | |
| Ki-67 | Invitrogen | 14-5698-82 | | 1:200 | |
| IL-13Rα2 | R&D | AF146 | | | 1:250 |
| Ki-67 | Dako | GA626 | | | 1:400 |
| NeuN | Sigma | MAB377 | | | 1:2400 |
| CD68 | Abcam | Ab125212 | | | 1:800 |

### Cell Proliferation and Viability Assays

Cells in single-cell suspension were plated with culture medium in 96-well clear bottom black microplates (Cat #3917; Corning Costar, Corning, NY, USA) at a density of 2,500 cells per well for adult GBM cell lines (GBM6, GBM 10, GBM14, GBM 39, GBM43, and GBM 108) or 5,000 cells per well for DMG cell lines (SU-DIPG XIII-P, SU-DIPG XVII, SF8628, SF8628-B23, and PED17) and cultured overnight at 37° C. with 5% CO2. The next day, cells were treated in triplicate with either vehicle (ddH20 or PBS) or serial dilutions of IL13 (to final concentrations of 100 ng/ml, 50 ng/ml, 20 ng/ml, 10 ng/ml, 5 ng/ml, 1 ng/ml, and 0.5 ng/ml) or cmIL13 (to final concentrations of 320 ng/ml, 100 ng/ml, 32 ng/ml, 10 ng/ml, 3.2 ng/ml, 1 ng/ml, 0.32 ng/ml, 0.1 ng/ml, 0.032 ng/ml, 0.01 ng/ml, 0.0032 ng/ml, and 0.001 ng/ml). Cells were incubated for 72 h then assayed with CellTiter-Glo Luminescent Cell Viability Assay (Cat #G7570; Promega, Madison, WI, USA) according to the manufacturer's recommendations. Luminescence was measured using an Infinite M200 PRO multimode microplate reader (Tecan Group, Männedorf, Switzerland), normalized to control wells (ddH20 or PBS only), and relative luminescence treatment was plotted as a function of drug concentration. The potency (50% inhibitory concentration, IC₅₀) of each treatment was calculated by nonlinear least-squares curve-fitting using Prism 9 (GraphPad, San Diego, CA, USA).

### Immunofluorescence

Cells were plated in single-cell suspension at a density of 10,000 cells per well on 4 Chamber Cell Culture Slides (Cat #50-114-9053; CELLTREAT Scientific Products, Pepperell, MA, USA) and cultured overnight at 37° C. with 5% CO2. After 24 h, cells were treated with either vehicle (PBS) or the IC₅₀ concentration of cmIL13, as determined by CellTiter-Glo Luminescent Cell Viability Assay (Promega). At specific timepoints (8 h, 24 h, 48 h, and 72 h), cells were then washed in PBS and fixed with 4% paraformaldehyde for 20 minutes. Cells were washed 3 times for 5 minutes each in PBS and incubated in 0.5% Triton X-100 in PBS for 5 minutes. To wash the coverslips of the permeabilization buffer, cells were incubated in PBS 3 times for 5 minutes each before blocking with 3% BSA in PBS-T for 1 hour at room temperature. Up to two different primary antibodies were then added in 1% BSA in PBST overnight at 4° C. Dilution buffer was used in lieu of primary antibody for cell-specific negative controls. The next day, cells were washed 3 times for 5 minutes each in PBS-T. Cells were then incubated with Alexa Fluor-coupled secondary antibodies (Thermo Fisher Scientific) in 1% BSA in PBS-T for 1 hour at room temperature in the dark. To test for cross-reactivity, one control per primary antibody condition was included by applying the other secondary antibody to the primary antibody. After 3 additional 5-minutes washes in PBS, chambers were removed and slides were rinsed thrice in dH20. Slides were mounted using ProLong Gold Antifade reagent with DAPI (Cat #P36935; Thermo Fisher Scientific) and stored at 37° C. until microscopy imaging. All slides were examined, and images captured using a LSM 780 confocal laser scanning microscope (Carl Zeiss Microscopy, White Plains, NY, USA). Detailed information regarding antibodies used for immunofluorescence can be found in Table 3, above.

### Patient-Derived Xenografts

All animal experiments were conducted in accordance with the NIH and IACUC guidelines for the use of animals in research and approved by the Mayo Clinic Institutional Committee for Animal Research. HGG cell lines (GBM6, PED17, and SU-DIPG XIII-P) were transduced with a luciferase reporter system (eGFP/fLuc2) that allows bioluminescence readout of tumor volume. Orthotopic tumor inoculation with cultured cells was performed as previously described in Welby J P, Kaptzan T, Wohl A, Peterson T E, Raghunathan A, Brown D A, Gupta S K, Zhang L, Daniels D J (2019) Current Murine Models and New Developments in H3K27M Diffuse Midline Gliomas. Front Oncol 9:92 and Carlson B L, Pokorny J L, Schroeder M A, Sarkaria J N (2011) Establishment, maintenance and in vitro and in vivo applications of primary human glioblastoma multiforme (GBM) xenograft models for translational biology studies and drug discovery. Curr Protoc Pharmacol Chapter 14: Unit 14.16 incorporated herein by reference. Briefly, cells were placed in single-cell suspension, and 300,000 cells in 3 μl of sterile PBS were prepared for engraftment into each mouse. A 0.5 mm burr hole was created at the following coordinates: 1 mm posterior and 2 mm to the right of the bregma (GBM6) or 1 mm posterior to the lambdoid suture and 1 mm lateral to the mid-sagittal plane (PED17 and SU-DIPGXIII). Using a 26-gauge (51 mm, point style AS) syringe (Cat. #203185; Hamilton Company. Bonaduz, Switzerland), tumor cells were injected stereotactically at a constant flow rate of 0.5 l/min into the cerebral hemisphere (GBM6) or pons (PED17 and SU-DIPGXIII) of 6 to 7 week-old female Hsd: Athymic Nude-Foxn 1^{nu} mutant mice that were obtained from Envigo (Madison, WI, USA). The injection depth was 4 mm for all groups. In vivo tumor engraftment and progression was monitored by bioluminescence imaging (BLI). Animals were dosed with an intraperitoneal injection of 10 mg/kg of Cycluc. After 10 minutes, mice were imaged under isoflurane anesthesia using an IVIS-200 Imaging System (Xenogen Corporation, Berkeley, CA). Image analysis was performed using LivingImage 4.3 (PerkinElmer, Waltham, MA, USA) to quantitate total flux (number of photons per second) within a region of interest.

For brain-targeted drug delivery, animals were randomized to control (PBS) and treatment (cmIL13) groups based on BLI signal to ensure equal distribution of tumor sizes at the beginning of the study (when BLI reached approximately 1,000,000 total log flux). Mice were placed under anesthesia with 100 mg/kg of ketamine and 10 mg/kg of xylazine. A 2-cm midline skin incision was made extending from behind the eyes to level of the ears. The previously established burr hole was reopened, and mice were secured on a stereotactic stage with automated thermal support using a Rodent Warmer X1 (Cat #53800M; Stoelting, Wood Dale, IL, USA). A 33-gauge internal cannula (Cat #8IC315IS5SPC; P1 Technologies, Roanoke, VA), with a 4 mm projection below the pedestal, was inserted into a 26-gauge guide cannula (Cat #8IC315GS5SPC, P1 Technologies), with a 3.5 mm projection below the pedestal, and both were connected to PE tubing and secured with a single connector-assembly (#C313C/SPC; P1 Technologies). The whole unit was secured vertically with a cannula holder (Cat #505254; World Precision Instruments, Sarasota, FL, USA) and connected to a 22-gauge (51 mm, point style AS) syringe (Cat #80400; Hamilton Company) placed in a Legato 130 syringe pump (Cat #788130; KD Scientific. Holliston, MA, USA). Vehicle (PBS) and drug (cmIL13 at concentrations of 50 μg/ml (1 μg dose), 15 μg/ml (0.3 μg dose), or 5 μg/ml (0.1 μg dose)) solutions were subsequently primed through the internal cannula and associated tubing. The cannula holder with attached internal cannula was lowered until flush with the mouse skull to reach the desired injection depth of 4 mm (GBM6) or 4.2 mm (PED17 and SU-DIPGXIII). In all study groups, the same ramped CED infusion protocol was performed with a total volume infused of 20 μl and rates of infusion as follows: 3 μl at 0.2 l/min, 5 μl at 0.5 μl/min, and 12 μl at 0.8 μl/min. To avoid reflux into the injection tract, the cannula was removed 10 min after completion of infusion. Animals were monitored daily and euthanized at indication of progressive neurologic deficit or if found in a moribund condition.

### Immunohistochemistry

Following animal euthanasia by carbon dioxide inhalation, brains were harvested and fixed in 4% paraformaldehyde at room temperature overnight. The brains were then embedded in paraffin and sectioned in the coronal plane (5 μm/section) using a microtome (CM1860 UV; Leica Biosystems, Buffalo Grove. IL. USA). Hematoxylin and eosin (H&E) staining was performed according to standard procedures. For immunohistochemistry, paraffin-embedded tissue sections were dewaxed in xylene and rehydrated in ethanol. Antigen retrieval was performed by steaming slides in preheated sodium citrate buffer (10 mM tri-sodium citrate, 0.05% Tween 20, pH 6.0) for 30 minutes. Sections were cooled to room temperature and rinsed with dH20 for 1 minute. This was followed by soaking sections in 0.6% hydrogen peroxide in MeOH for 20 minutes. Sections were then blocked with 10% normal goat serum (NGS) in Tris-buffered saline (TBS) for 30 min at room temperature. Primary antibodies were diluted in TBS with 2% NGS and 0.5% Triton X-100 and applied to sections overnight at 4° C. Dilution buffer was used instead of primary antibody for tissue-specific negative controls. The next day, sections were washed 3 times for 5 minutes in TBS with 2% NGS and 0.5% Triton X-100. The VECTASTAIN Elite ABC kit (Cat #PK-6100; VECTOR Laboratory, Burlingame, CA) containing biotinylated secondary antibody was diluted in TBS with 1.5% NGS and added to the sections according to manufacturer's recommendations. After 3 additional 5-minutes washes in TBS, sections were incubated with Avidin/Biotinylated Enzyme Complex (ABC) solution (Cat #PK-6100; VECTOR Laboratory) for 30 minutes at room temperature. For visualization, the sections were subsequently developed using SignalStain DAB Substrate Kit (Cat #8059P; Cell Signaling, Danvers, MA, USA) per the manufacturer's protocol, counterstained with hematoxylin, and mounted with permount (Cat #SP15-100, Thermo Fisher Scientific). Images were acquired with a digital slide scanner (Axio Scan.Z1; Carl Zeiss Microscopy) and are presented at a magnification of 40×. Detailed information regarding antibodies used for immunohistochemistry (IHC) is provided in Table 3, above. Low-power images were included to demonstrate consistency of staining in tissue sections.

### Statistical Analyses

The data were collected and presented as mean #standard deviation or standard error of the mean when appropriate. Direct statistical comparisons between 2 groups were conducted using two-tailed Student's t-tests. Nonlinear least-squares curve-fitting analysis was used to determine the potency (IC₅₀) of cmIL13 treatment in vitro. Survival analysis was performed using the Kaplan-Meier estimate with Log-Rank test. Statistical tests and analyses were conducted using Prism 9 (GraphPad), with statistical significance set at an a threshold of 0.05, and p<0.05 marked by asterisks in figures.

### Results:

### IL-13Rα2 is Expressed at Different Levels in HGG Tumor Cell Models

To identify baseline transcription and protein levels of IL-13Rα2 in HGG cells,

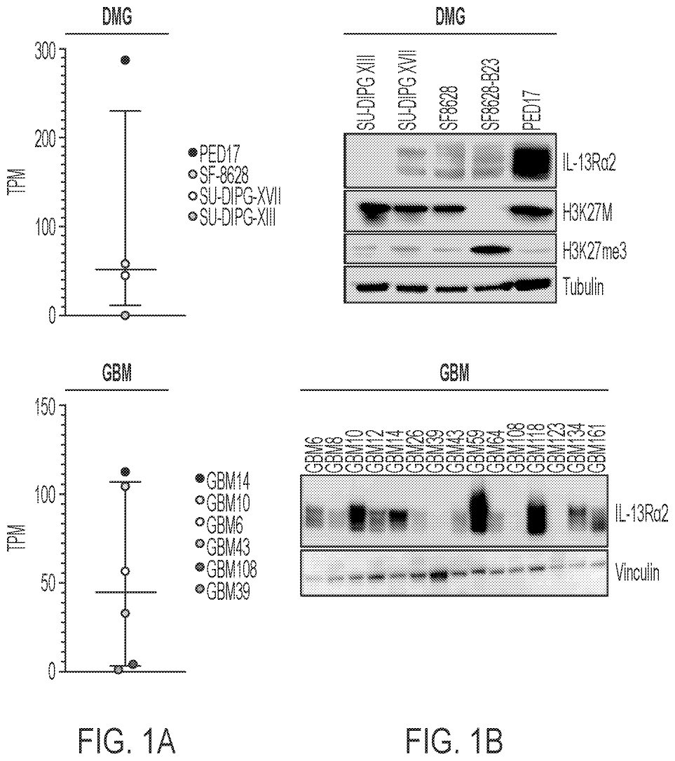

RNA sequencing and immunoblotting were performed on 10 patient-derived HGG (4 DMG and 6 adult GBM) cell lines (FIG. 1A, 1B). In accordance with previous investigations, the cohort of sequenced HGG transcriptomes confirmed the differential expression pattern of IL-13Rα2 RNA among HGG cell lines (FIG. 1A). IL-13Rα2 transcription levels in both DMG and adult GBM models ranged from low (SU-DIPG XIII-P, GBM39, GBM108) to medium (SU-DIPG XVII, SF8628, GBM43, GBM6) and strong (PED17, GBM10, GBM14) expression. Next, IL-13Rα2 protein levels were evaluated in available HGG cell lines. Correspondingly, IL-13Rα2 protein levels were congruent with gene expression in both DMG and adult GBM cell lines (FIG. 1B). Several cell models showed high IL-13Rα2 expression, including PED17, GBM10, GBM14, GBM59 and GBM118, while others, such as SU-DIPG XVII, SF8628, SF8628-B23, GBM6, GBM12 and GBM43, showed notably lower (but not absent) IL-13Rα2 levels. A third category of HGG cell lines, including SU-DIPG XIII-P, GBM39, GBM108 and GBM123, demonstrated IL-13Rα2 protein levels that were below the detection threshold of the assay.

### Functional Impact of IL13Rα2 on HGG Proliferation and Survival

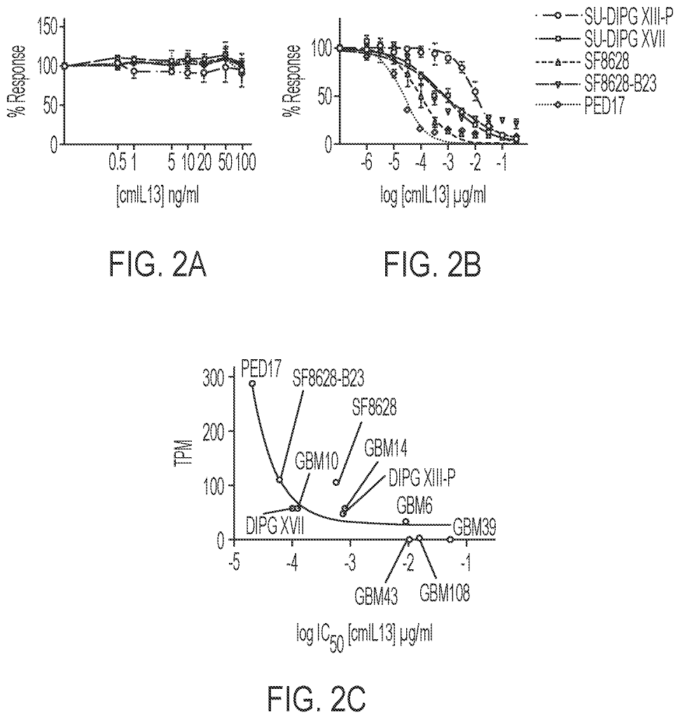

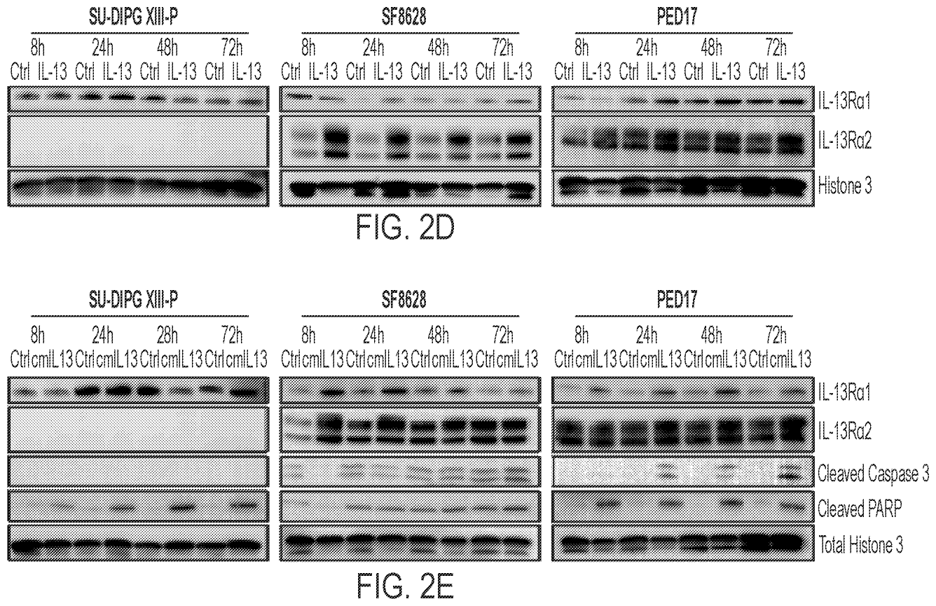

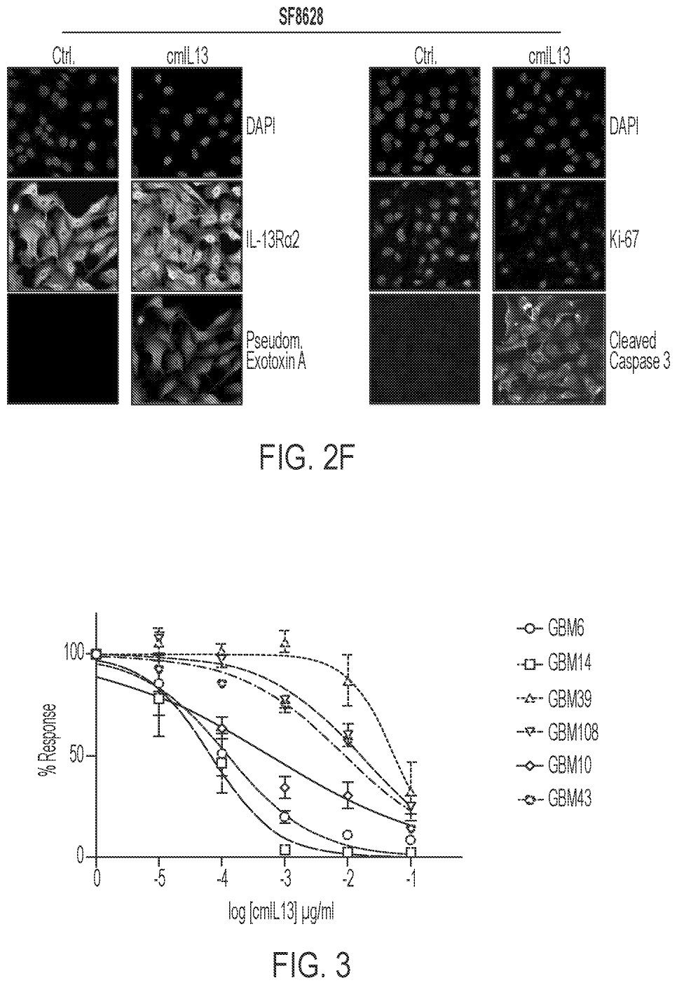

Given the cell line-dependent overexpression of IL-13Rα2 in the DMG and adult GBM tumor cell models, the role of IL-13Rα2 signaling in HGG was investigated (FIG. 2A-F). To determine whether cytokine stimulation impacts cell proliferation in vitro, HGG cells were treated with varying concentrations of the canonical ligand of IL-13Rα2, IL13. While the lack of SU-DIPG XIII-P response was consistent with the low expression of IL-13Rα2 in the assayed cell models, none of the IL-13Rα2-medium or IL-13Rα2-high cell lines stimulated with IL13 demonstrated any significant increase in cell proliferation versus media as the control (FIG. 2A). Based on previous reports, which showed that IL-13Rα2 is implicated in cell survival rather than cell growth and invasion, it was hypothesized that cytokine stimulation would be associated with increased IL-13Rα2 expression to enforce this anti-apoptotic effect. To test this, HGG cells were stimulated with IL-13 (10 ng/ml) and investigated protein levels at various time points (FIG. 2C). Stimulation with IL13 resulted in robust upregulation of IL-13Rα2 in IL-13Rα2-medium and IL-13Rα2-high cell lines after 8 h, 24 h, 48 h, and 72 h. Conversely, IL-13Rα1 levels remained unaffected by IL13 stimulation in all assayed HGG cell models.

cmIL13 Elicits Potent Anti-Tumor Effects in HGG Cell Models

To assess whether IL-13Rα2 expression confers sensitivity to IL-13Rα2-targeted therapy in vitro, the pharmacological response of HGG cells to cmIL13 was tested. Eleven HGG (5 DMG and 6 adult GBM) cell lines were selected and exposed to varying concentrations of cmIL13, with treatments ranging from 0.001 ng/ml-320 ng/ml. The results showed a direct relationship between IL-13Rα2 expression and cmIL13 sensitivity (FIG. 2B, FIG. 3). cmIL13 demonstrated strong cytotoxicity in IL-13Rα2-high cell lines, which was marked by a prominent left-shift on the dose response curve versus comparatively insensitive IL-13Rα2-low cell models. The IC₅₀ values of cmIL13 in IL-13Rα2-high cells were: 0.02 ng/ml for PED17 cells, 0.06 ng/ml for GBM14, and 0.58 ng/ml for GBM10. IL-13Rα2-medium cells displayed the following IC₅₀ values for cmIL13:0.10 ng/ml for SF8628, 0.75 ng/ml for SU-DIPG XVII, 0.81 ng/ml for SF8628-B23, 0.12 ng/ml for GBM6, and 9.08 ng/ml for GBM43. Finally, the IC₅₀ values for cmIL13 in IL-13Rα2-low cells were: 10.63 ng/ml for SU-DIPG XIII-P, 15.74 ng/ml for GBM108, and 53.82 ng/ml for GBM39. Both DMG and adult GBM cell models showed similar sensitivity towards cmIL13 dependent on IL-13Rα2 status.

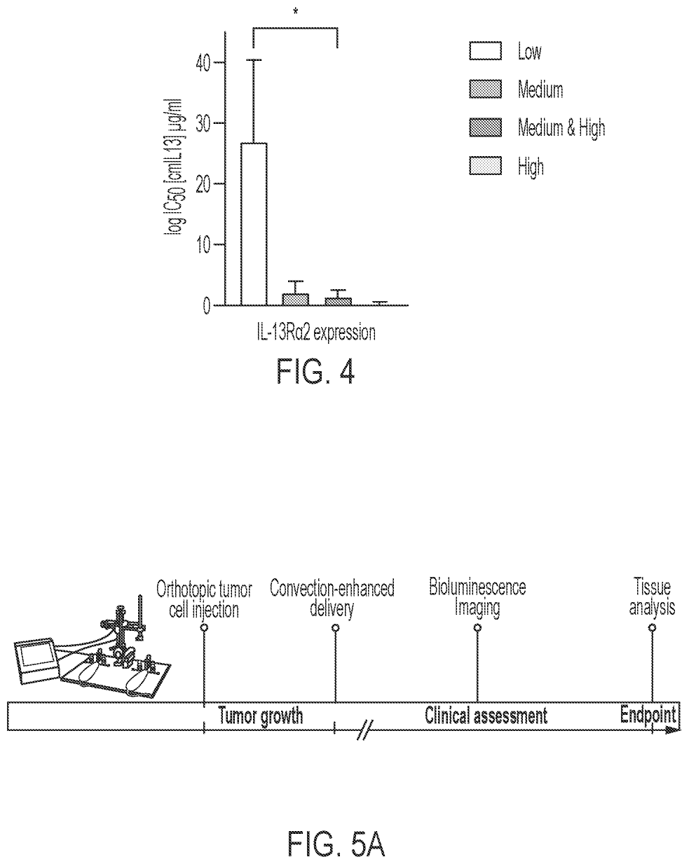

To gain insight into the effects of cmIL13 on IL-13Rα2, next HGG cells were treated with IC₅₀ concentrations of the drug and investigated protein levels at 8 h, 24 h, 48 h, and 72 h (FIG. 2D). Similar to IL13 stimulation, cmIL13 did not induce IL-13Rα2 downregulation but rather led to stable or increased protein levels over time. Intriguingly, IL-13Rα1 was upregulated in some IL-13Rα2-medium and IL-13Rα2-high cell models exposed to cmIL13. Furthermore, apoptosis induction was marked by increased levels of cleaved caspase 3 and/or cleaved PARP. These results were confirmed with confocal microscopy (FIG. 2E, FIG. 4), where prominent IL-13Rα2 levels were found at baseline, which were retained in cells treated with cmIL13 for up to 72 h. By staining for the PE-domain of cmIL13, colocalization of the drug to the receptor as well as internalization into the cytoplasm and nucleus was confirmed. In addition to increased levels of apoptosis, cellular proliferation was decreased in the presence of cmIL13.

Intratumoral Administration of cmIL13 Results in Decreased Tumor Burden and Prolonged Survival In Vivo

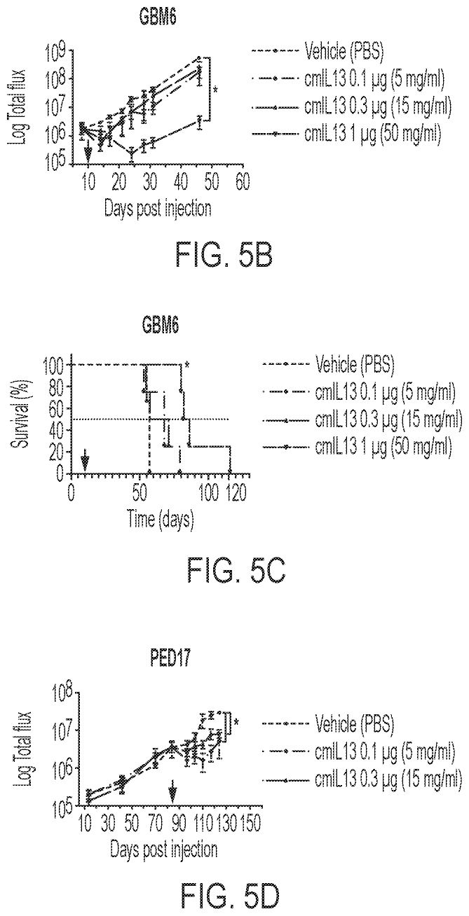

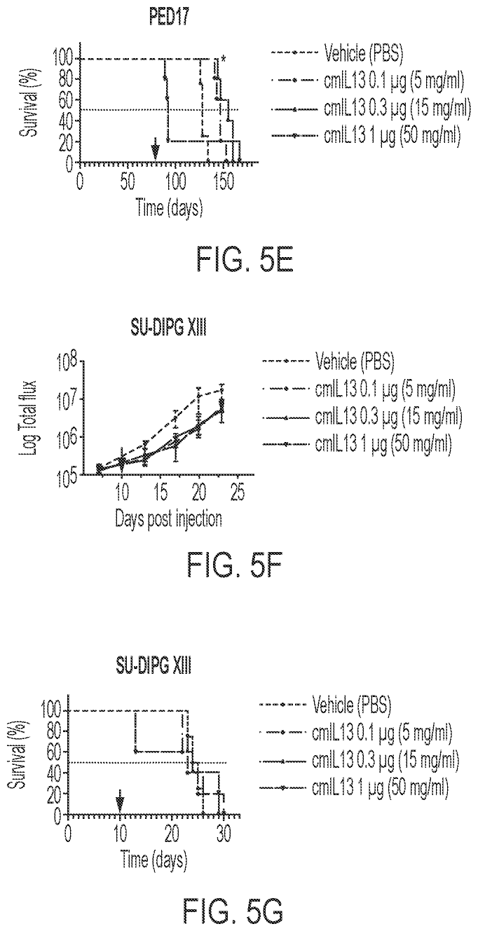

In order to validate the anti-tumor effects of emIL13 in vivo, orthotopic patient-derived murine xenograft models of HGG were utilized, including IL-13Rα2-low (SU-DIPG XIII-P), IL-13Rα2-medium (GBM6), and IL-13Rα2-high (PED17) models. Tumor-bearing animals were randomized into four cohorts and treated with a single, brain-targeted dose of cmIL13 via CED in 4-5 animals per treatment arm (FIG. 5A). A drug delivery system was initially established in adult GBM animals by infusing vehicle solution (PBS) or various doses of cmIL13 into the hemispheric GBM6 tumor region. All CED systems were placed and tolerated without complications. There were no procedure-related deaths, and clinical assessments of animals after completed infusions were all unremarkable with no signs of acute or delayed toxicities or neurological deficits. The tumor volume, measured by BLI, was significantly lower in animals treated with 1 μg of cmIL13 (p=0.01) as compared to the 0.3 μg (p=0.14), 0.1 μg (p=0.08) and vehicle-treated groups (FIG. 5B). A single dose of 1 μg cmIL13 significantly prolonged survival with a median survival of 84 days (p=0.01) in comparison to 64 days in 0.3 μg cmIL13 (p=0.35), 68 days in 0.1 μg cmIL13 (p=0.17) and 57 days in vehicle groups (FIG. 5C).

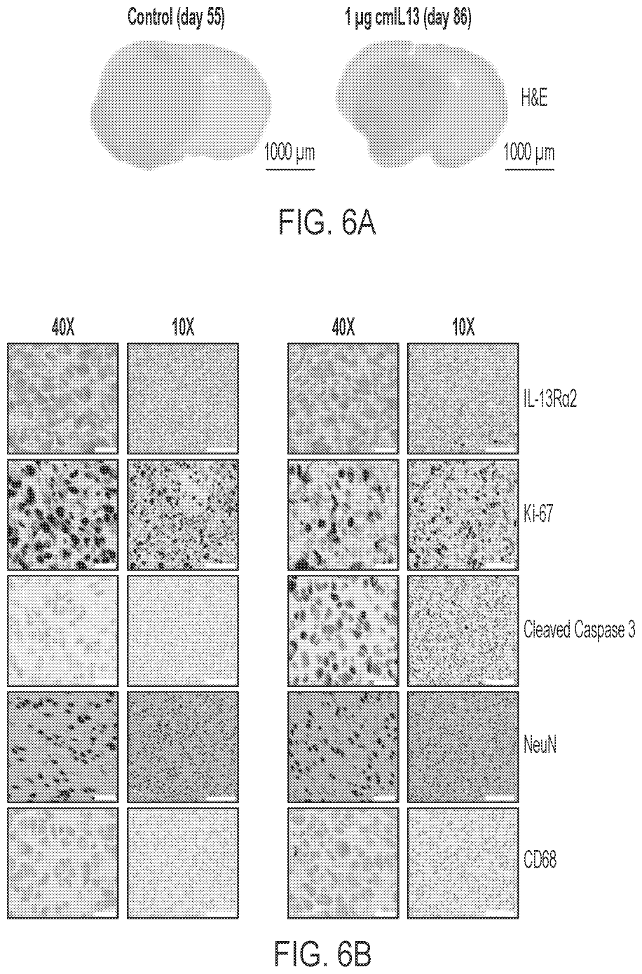

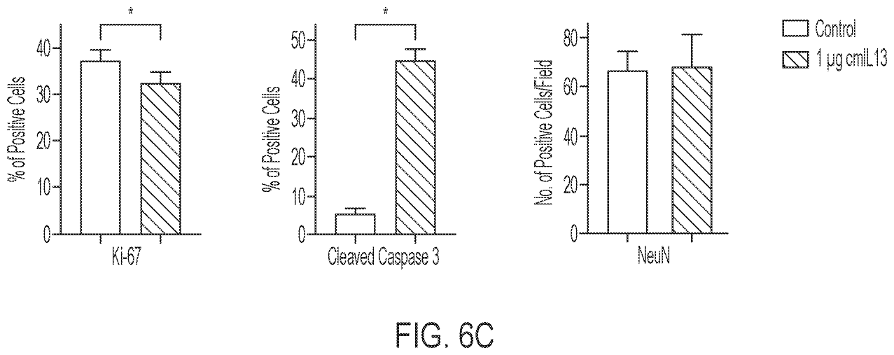

Histologic evaluation of brains from mice euthanized in a moribund state demonstrated maintained tissue architecture and decreased tumor size after cmIL13 treatment (FIG. 6A). On-target drug effects were validated in tumors by IHC analysis of IL-13Rα2 levels, apoptosis induction, and cellular proliferation in drug-treated mice compared to control. In agreement with the in vitro data, high IL-13Rα2 status was retained in GBM6 cells (FIG. 6B). Cellular proliferation, which was determined by Ki-67 staining, was decreased following exposure to cmIL13 (FIGS. 6B and 6C). Intriguingly, intense staining for the apoptosis marker cleaved caspase 3 was evidenced throughout the tumor area in all cmIL13 groups but absent in vehicle-treated animals weeks after cmIL13 administration (FIG. 6B). To address toxicity considerations that may accompany immunotoxin delivery into the brain, additional IHC analyses for NeuN were performed, a marker of mature neurons, and CD68, which is expressed in high levels by microglia and monocytes. CED of cmIL13 did not result in a decrease of NeuN-positive cells in the infused, ipsilateral hemisphere as compared to vehicle (FIGS. 6B and 6C). No immune cell infiltration was evidenced in any study group (FIGS. 6B and 6C).

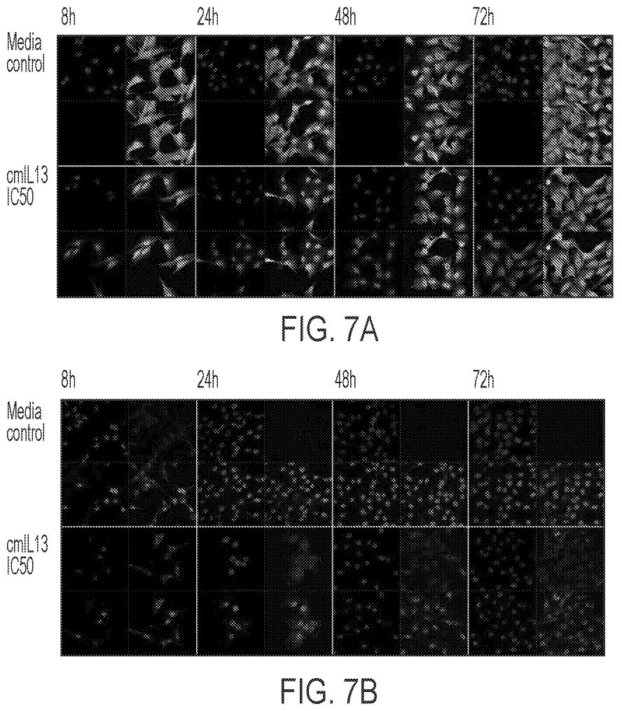

These findings were validated in an IL-13Rα2-upregulated DMG xenograft model. PED17 cells were orthotopically implanted into the pons, and tumor-bearing animals were again treated with vehicle solution. 0.1 μg, 0.3 μg, or 1 μg of cmIL13. In line with prior observations, all animals tolerated the CED procedure; however, at the highest dose (a 1 μg CED infusion of cmIL13), marked signs of toxicity developed in 5/5 animals within 24 h of infusion (neurological deficits such as hemiparesis or ataxia, hunched body position, dermatitis), and 4/5 animals had to be euthanized within 72 h of drug administration. Post-operative clinical assessments were unremarkable for animals treated with 0.1 μg or 0.3 μg of cmIL13. Comparison of BLI signal demonstrated that a single 0.1 μg or 0.3 μg cmIL13 infusion significantly decreased tumor volume (p=0.0001 and 0.0004, respectively; FIG. 5D) and significantly extended median survival (147 days for 0.1 μg cmIL3 (p=0.003) and 155 days for 0.3 μg cmIL13 (p=0.003)) compared to the vehicle group (128 days) (FIG. 5E). Similar to the findings in hemispheric GBM6 tumors, none of the 0.1 μg or 0.3 μg doses had an impact on NeuN+ cell density and CD68+ cell infiltration. Consistent with the observed differential in clinical toxicity, a 1 μg dose of cmIL13 resulted in a marked decrease in NeuN-positive cells in the brainstem as compared to lower cmIL13 doses or vehicle control. There was no evidence of monocyte cell infiltration following exposure to 1 μg cmIL13. Detected levels of IL-13Rα2 remained constant among treatment groups. A decrease in DMG-characteristic H3 K27M and increase in H3 K27me3 was evidenced in drug treated tumors. Additional IHC findings equally paralleled the results of the first study (FIGS. 7A and 7B).

Finally, the DMG cell line SU-DIPG XIII-P was used to establish a HGG xenograft model with low IL-13Rα2 protein levels. Based on the in vitro data, it was not expected for cmIL13 to impact tumor volume or survival using the previously established dosing regimen. Indeed, CED of 0.1 μg, 0.3 μg, or 1 μg of cmIL13 failed to demonstrate significant tumor growth reduction (p=0.16, 0.18 and 0.27, respectively; FIG. 5F) and did not provide profound survival benefit compared to control animals (24.5 days in vehicle, 23 days in 0.1 μg cmIL13 (p=0.92), 24 days in 0.3 μg cmIL13 (p=0.68) and 24 days in 1 μg cmIL13 (p=0.57)) (FIG. 5G). While the CED procedure proved to be feasible and safe among all treatment groups, and no immune cell infiltration or reduction in NeuN-positive cells was observed in mice treated with 0.1 μg or 0.3 μg of cmIL13, there was again evidence of reduced NeuN+ cell density in the brainstem of animals treated with 1 μg of cmIL13. IHC did not show increased staining for cleaved caspase 3 in cmIL13 drug-treated IL-13Rα2-low xenografts, and a high degree of cellular proliferation was retained in these tumors after cmIL13 infusion. In accordance with in vitro protein-level analysis, IHC staining for IL-13Rα2 was absent in SU-DIPG XIII-P xenografts. Furthermore, H3 K27M and H3 K27me3 remained largely unchanged in vehicle versus cmIL13 treated tumors. These results indicate that IL-13Rα2 upregulation is required for targeted therapies such as cmIL13 to impart therapeutic effect in HGG orthotopic xenograft models.

Whereas specific aspects of the disclosure have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the disclosure which is to be given the full breadth of the claims and aspects appended and any and all equivalents thereof.

We claim:

1. A method of treating a subject having a malignant glioma comprising:
administering by convection-enhanced delivery (CED) for a period of up to 96 hours a therapeutically effective amount of a mutagenized IL13 (mIL13) linked to a cytotoxin (cmIL13);
wherein the malignant glioma expresses an interleukin 13 receptor α 2 (IL13Rα2).

2. The method of claim 1 wherein the cmIL13 is administered for a period of 4 hours to 96 hours, at a dose of 0.03 μg/mL to 1 μg/mL, and/or at a flow rate of up to 1 mL/hour.

3. (canceled)

4. (canceled)

5. The method of claim 1, wherein the cytotoxin comprises a bacterial-derived toxin.

6. The method of claim 5, wherein the bacterial-derived toxin comprises Pseudomonas exotoxin A.

7. (canceled)

8. (canceled)

9. The method of claim 1, further comprising co-infusing an imaging component.

10. The method of claim 1, further comprising detecting expression of IL13Rα2 in a sample, wherein expression of the IL13Rα2 is indicative of the malignant glioma being responsive to treatment by cmIL13.

11. The method of claim 10, wherein the sample comprises:
a liquid biopsy of blood, plasma, saliva, urine, and/or cerebral spinal fluid; and/or
a solid biopsy of an organ and/or a tissue.

12. (canceled)

13. (canceled)

14. (canceled)

15. The method of claim 10, wherein detecting expression of IL13Rα2 is done by mass spectrometry, analytical assay, immunostaining, and/or sequencing.

16. (canceled)

17. The method of claim 1, wherein the administering results in an increase in survival of the subject relative to a control subject having a malignant glioma that is not administered a therapeutically effective amount of the cmIL13.

18. (canceled)

19. The method of claim 1, wherein the method results in no detectible changes in clinically relevant biomarkers within non-glioma cells and/or non-glioma tissue.

20. The method of claim 19, wherein the biomarkers comprise NeuN and CD68+ proteins.

21. The method of claim 1, wherein the administration of the cmIL13 to cells expressing IL13Rα2 results in:
(i) a reduction in volume of the malignant glioma;
(ii) an increase in detectable cleaved caspase 3, as measured by IHC or fluorescent cytochemistry; and/or
(iii) a reduction in the level of Ki-67-positive cells by an amount of at least 10%, as measured by IHC or fluorescent cytochemistry.

22. The method of claim 21, wherein the reduction in volume of the malignant glioma is determined by an Analysis of Variants (ANOVA) test, wherein statistical significance is determined by a p value of <0.05.

23. The method of claim 21, wherein the reduction in volume of the malignant glioma is determined by a two-tailed Student's t-test, wherein statistical significance is determined by a p value<0.05.

24. The method of claim 1, wherein the mIL13 is engineered to have increased affinity for IL13Rα2 compared to native human IL13 and/or decreased affinity for interleukin 13 receptor α 1 (IL13Rα1) compared to native human IL13.

25. The method of claim 1, wherein the m13 is characterized by at least one amino acid substitution as compared to SEQ ID NO:1 or SEQ ID NO:2.

26. The method of claim 1, wherein the mIL13 comprises amino acid changes relative to wild type IL13 at positions E13, R66, S69, and/or K105.

27. The method of claim 1, wherein the mIL13 comprises one or more amino acid substituents E13K.R66D.S69D.K105R.

28. The method of claim 1, wherein the mIL13 comprises an amino acid sequence set forth in one of SEQ ID NOS: 3 to 24 or a homologue thereof or a homologue of one of SEQ ID NO:1 or SEQ ID NO:2.

29. The method of claim 1, wherein the mIL13 differs by no more than 20 residues from SEQ ID NO:1 or SEQ ID NO:2.

30. (canceled)


SEQUENCE LISTING (24 sequences)

SEQ ID NO: 1 - protein, 146 aa, Homo sapiens [sequence omitted]

SEQ ID NO: 2 - protein, 113 aa, Homo sapiens
PGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 3 - protein, 114 aa, Homo sapiens
SPGPVPPSTALRKLIEELVNITQNQKAPLCNGSMVWSINLTAGMYCAALESLINVSGCSAIEKTQDMLDGFCPHKVSAGQFSSLHVRDTKIEVAQFVKDLLLHLRKLFREGRFN

SEQ ID NO: 4 - protein, 113 aa, Homo sapiens
PGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFN

SEQ ID NO: 5 - protein, 113 aa, Homo sapiens
PGPVPPSTAIRELIEELINITQNQKAPLCNGSMVWSINLTAGMYCAALESLINVSGCSAIEKTQRMLSGFCPHKVSAGQFSSLHVRGSKIEVAQFVKDLLHHLRALFREGQFN

SEQ ID NO: 6 - protein, 113 aa, Homo sapiens
PGPVPPSTAVRELIEELINITQNQKAPLCNGSMVWSINRTAGMYCAALESLINVSGCSAIEKTQRMLSGFCPHKVSAGQFSSLHVRSSKIEVAQFVKDLLFHLRTLFREGQFN

SEQ ID NO: 7 - protein, 113 aa, Homo sapiens
PGPVPPSTALIELIEELINITQNQKAPLCNGSMVWSINLTAGIYCAALESLINVSGCSAIEKTQRMLSGFCPHKVSAGQFSSLHVKGSKIEVAQFVKDLLHHLRALMREGQFN

SEQ ID NO: 8 - protein, 113 aa, Homo sapiens
PGPVPPSTAIRELIEELLNITQNQKAPLCNGSMVWSINLTAGMYCAALESLINVSGCSAIEKTQRMLSGFCPHKVSAGQFSSLHVMKSKIEVAQFVKDLLHHLRALFREGQFN

SEQ ID NO: 9 - protein, 113 aa, Homo sapiens
PGPVPPSTAIRELIEELINITQNQKAPLCNGSMVWSINLTAGMYCAALESLINVSGCSAIEKTQRMLSGFCPHKVSAGQFSSLHVRSSRIEVAQFVKDLLHHLRTLFREGQFN

SEQ ID NO: 10 - protein, 113 aa, Homo sapiens
PGPVPPSTALRELIEELINITQNEKAPLCNGSMVWSINLTAGIYCAALESLINVSGCSAIEKTQRMLSGFCPHKVSAGQFSSLHVTGSKIEVAQFVKDLLYHLRALFREGQFN

SEQ ID NO: 11 - protein, 113 aa, Homo sapiens
PGPVPPSTALSELIEELINITQNQKAPLCNGSMVWSINPTAGMYCAALESLINVSGCSAIEKTQRMLSGFCPHKVAAGQFSSLHDKGSMIEVAQFVKDLLYHLRTLFREGQFN

SEQ ID NO: 12 - protein, 113 aa, Homo sapiens
PGPVPPSTATRELIEELINITQNQKAPLCNGSMVWSINLTADMYCAALESLINVSGCSAIEKTQRMLSGFCPHKVSVGQFSSLHVRGSKIEVAQFVKDLLYHLRTLFREGQFN

SEQ ID NO: 13 - protein, 113 aa, Homo sapiens
PGPVPPSTADIELIAELINITQNQKAPLCNGSMVWSINLTADMYCAALESLINVSGCSAIEKTQRMLSGFCPHKVSAGQFSSLHVKKTRIEVAQFVKDLLLHLKKLFKEGQFN

SEQ ID NO: 14 - protein, 113 aa, Homo sapiens
PGPVPPSTAARELIEELVNITQNQKAPLCNGSMVWSINLTAGMYCAALESLINVSGCSAIEKTQRMLSGFCPHKVSAGQLSSLHVTGKRIEVAQFVKDLLNHLRALFKEGQFN

SEQ ID NO: 15 - protein, 113 aa, Homo sapiens
PGPVPPSTAVRELIEELVNITQNQKAPLCNGSMVWSINLTAGMYCAALESLINVSGCSAIEKTQRMLSGFCPHKVSAGQFSSLHVRDTRIEVAQFVKDLLNHLKELFTEGQFN

SEQ ID NO: 16 - protein, 113 aa, Homo sapiens
PGPVPPSTALSELMEELVNITQNQKAPLCNGSMVWSINLTAGMYCAALESLINVSGCSAIEKTQRMLSGFCPHKVSAGQFSSLHVRDSKIEVAQFVKDLLNHLKALFKEGQFN

SEQ ID NO: 17 - protein, 112 aa, Homo sapiens
GPVPPSTAFRELIEELVNITQNQKAPLCNGSMVWSINLTAGMYCAALESLINVSGCSAIEKTQRMLSGFCPHKVSPGQFSSLHVTNSRIEVAQFVKDLLNHLKALFKEGQYN

SEQ ID NO: 18 - protein, 112 aa, Homo sapiens
GPVPPSTAHLELIEELINITQNQKAPLCNGSMVWSINLTAGMYCAALESLINVSGCSAIEKTQRMLSGFCPHKVSAGQFSSLHVKETRIEVAQFVKDLLNHLKTLFKEGQFN

SEQ ID NO: 19 - protein, 113 aa, Homo sapiens
PGPVPPSTAHLELIEELINITQNQKAPLCNGSMVWSINPTAGMYCAALESLINVSGCSAIEKTQRMLSGFCPHKVSAGQFSSLHVMDTRIEVAQFVKDLLLHLKKLFKEGQFN

SEQ ID NO: 20 - protein, 113 aa, Homo sapiens
PGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 21 - protein, 113 aa, Homo sapiens
PGPVPPSTAHRELIEELVNITQNQKAPLCNGSMVWRINRTAGMYCAALESLINVSGCSAIEKTQRMLSGFCPHKVSAGQFSSLHVMDSRIEVAQFVKDLLNHLRALFKEGQFN

SEQ ID NO: 22 - protein, 113 aa, Homo sapiens
PGPVPPSTAARELIEELFNITQNQKAPLCNGSMVWSINLTAGMYCAALESLINVSGCSAIEKTKRMLSGFCPHKVSAGQFPSLHVKKTRIEVAQFVKDLLIHLRKLFKEGQFN

SEQ ID NO: 23 - protein, 81 aa, Homo sapiens
MVWSINLTAGMYCAALESLINVSGCSAIEKTQRMLSGFCPHKVSAGQFSSLHVRDTKIEVAQFVKDLLLHLKKLFREGQFN

SEQ ID NO: 24 - protein, 146 aa, Homo sapiens
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN